(19) United States
(12) Patent Application Publication
(10) Pub. No.: US 2014/0162903 A1
(43) Pub. Date: Jun. 12, 2014

(54) Metabolite Biomarkers For Forecasting The Outcome of Preoperative Chemotherapy For Breast Cancer Treatment

(71) Applicant: Purdue Research Foundation, West Lafayette, IN (US)

(72) Inventors: M. Daniel Raftery, Seattle, WA (US); Siwei Wei, West Lafayette, IN (US); Susan E. Clare, Chicago, IL (US)

(73) Assignee: Purdue Research Foundation, West Lafayette, IN (US)

(21) Appl. No.: 14/068,923

(22) Filed: Oct. 31, 2013

Related U.S. Application Data
(60) Provisional application No. 61/720,644, filed on Oct. 31, 2012.

(51) Int. Cl. G01N 33/49

(52) U.S. Cl. CPC G01N 33/492
USPC 506/12; 506/15

(57) ABSTRACT

The present disclosure relates to a panel of metabolite species that is useful for forecasting the outcome of preoperative chemotherapy for the treatment of breast cancer, including methods for identifying and using such metabolite species that can be measured in biological samples taken before treatment. In preferred embodiments, a method of forecasting a treatment outcome before subjecting a breast cancer patient to preoperative chemotherapy is disclosed that includes measuring the concentration of at least one metabolite species in a sample of a biofluid taken from the breast cancer patient before preoperative chemotherapy treatment.

### CROSS-REFERENCE TO RELATED APPLICATIONS

This application claims benefit of U.S. Provisional Patent Application 61/720,644, filed Oct. 31, 2012, the entire contents of which are incorporated by reference for all purposes.

### TECHNICAL FIELD

The present disclosure generally relates to small molecule metabolic biomarkers. In particular, the present disclosure relates to a panel of metabolite species that is useful for forecasting the outcome of preoperative chemotherapy treatment for breast cancer, including methods for identifying such metabolic biomarkers within pretreatment biological samples.

### BACKGROUND

Breast cancer, although histologically similar, is clinically a very heterogeneous disease, which results in a range of treatment effectiveness and outcomes. Neoadjuvant, or preoperative, chemotherapy can significantly benefit some breast cancer patients. However, the varied responses to preoperative chemotherapy and resulting different outcomes mean that a significant proportion of the patient population is subjected to ineffective treatment while at the same time being exposed to the therapy's toxicities. Pathologic complete response (pCR), defined as the disappearance of the invasive cancer cells in the breast after chemotherapy, is used to evaluate patient response and is strongly associated with improved long-term survival rates. Unfortunately, less than 30% of patients overall show the outcome of pathological complete response to preoperative chemotherapy. An ability to predict the outcome of preoperative chemotherapy followed by surgery would enable development of personalized treatment protocols, improving survival rates and reducing unnecessary exposure of patients to toxic drugs.

Research focused on finding useful molecular or clinical predictors of pathologic complete response to neoadjuvant chemotherapy in breast cancer is relatively sparse. Imaging studies, such as magnetic resonance imaging (MRI) and scintimammography, were proposed to predict pathological responses to preoperative chemotherapy, but they are somewhat limited by low sensitivity combined with high costs. High levels of MUC-1 antigen (CA 15.3) in pretreatment serum and its fall after chemotherapy can predict responses as well, but many patients do not exhibit elevation of this marker before treatment and hence it is not helpful for such patients. Approaches using genomics and immunohistochemistry have been explored to find serum and tissue biomarkers. It has been shown that gene signatures such as Her2/neu over-expression/amplification and estrogen receptor (ER) expression were associated with pathologic complete response and certain preoperative chemotherapy regimens. Other molecular markers such as tumor RNA, glucose-regulated protein (GRP78) and hormone receptors have also been identified as potential predictors of pathologic complete response. However, suboptimal performance is a major issue that limits their wide applicability. Circulating tumor cells (CTC) have also been established as providing outcome predictions from particular therapies; however CTCs can be detected in less than 30% of early stage breast cancer patients, which limits its clinical applicability.

As an alternative approach for biomarker discovery, metabolomics (or metabolite profiling) enables identification of small-molecule metabolite species in biofluids and tissues that are sensitive to altered pathology. High-throughput analytical techniques of nuclear magnetic resonance (NMR) spectroscopy and mass spectrometry (MS) combined with multivariate statistical analyses provide information on a large number of metabolite species, including those that have altered levels between healthy subjects and patients with various diseases including cancer. So far, the metabolomics based approaches have been used in a large variety of applications, including early disease detection, drug response, toxicity and nutritional studies, and basic systems biology. Compared with other biomarker discovery approaches for breast cancer, metabolomics provides a strong link between genotype and phenotype, and may provide some insight into oncogenesis. Also, once established, tests based on metabolic profiles are relatively inexpensive, rapid and automated.

### SUMMARY OF THE INVENTION

The present disclosure relates to a panel of metabolite species that is useful for forecasting the outcome of preoperative chemotherapy for the treatment of breast cancer, including methods for identifying and using such metabolite species that can be measured in biological samples taken before treatment. In preferred embodiments, a method of forecasting a treatment outcome before subjecting a breast cancer patient to preoperative chemotherapy is disclosed that includes measuring the concentration of at least one metabolite species in a sample of a biofluid taken from the breast cancer patient before preoperative chemotherapy treatment.

In one aspect, the disclosure includes a method of forecasting a treatment outcome before subjecting a breast cancer patient to preoperative chemotherapy, comprising the steps of measuring the concentration of at least one metabolite species in a sample of a biofluid taken from a subject having breast cancer before preoperative chemotherapy treatment, wherein each metabolite species is a component of a panel of a plurality of metabolite species, wherein the components of the panel have been selected from a population of metabolite species that have been shown to significantly distinguish a first outcome from a second outcome; wherein a statistical model has been constructed using the combined measured concentration differences of each component in the panel of a plurality of metabolite species in a first sample from a subject having the first outcome and the measured concentration of each component of the panel of a plurality of metabolite species in a second sample from a subject having the second outcome, wherein the statistical model distinguishes the first sample from the second sample; using the statistical model to determine the relationship of the concentrations of the components of the panel of metabolite species measured in the test sample to the concentrations of the components of the panel of metabolite species that are characteristic of the first outcome and characteristic of the second outcome; thereby forecasting an outcome of subjecting a breast cancer patient to preoperative chemotherapy. In certain embodiments, the concentration of at least two metabolite species is measured.

In preferred embodiments, the outcomes being determined by the method are:
a. the first outcome is pathological complete recovery and the second outcome is stable disease, or
b. the first outcome is pathological complete recovery and the second outcome is partial response, or
c. the first outcome is stable disease and the second outcome is partial response; or
d. the first outcome is pathological complete recovery and the second outcome is stable disease plus partial response.

In certain embodiments, the method employs a panel of metabolite species comprises one to thirteen compounds selected from the group consisting of glutamine, histidine, isoleucine, threonine, 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine, and mixtures thereof. In some embodiments, the panel comprises one to five metabolite species selected from the group consisting of glutamine, histidine, isoleucine, threonine, linolenic acid and mixtures thereof. In other embodiments, the panel comprises linolenic acid.

In further embodiments, the method uses a panel of metabolite species consisting of components that have been selected from a population of metabolite species that have been shown to significantly distinguish a first outcome from a second outcome. Exemplary panels include, but are not limited to:
a. glutamine, histidine, isoleucine, threonine, and linolenic acid, or
b. glutamine, histidine, isoleucine, and threonine, or
c. glutamine, isoleucine, and threonine; or
d. glutamine, isoleucine, threonine, and linolenic acid; or
e. linolenic acid.

In other embodiments, the method uses a panel of metabolite species consisting of components that have been selected from a population of metabolite species that have been shown to significantly distinguish a first outcome from a second outcome. Exemplary panels include, but are not limited to:
a. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, 1-linoleoylphosphatidyl-choline, 1-hexadecyl-2-acetyl-glycero-3-phosphocholine, 2-amino-3-methyl-1-butanol, and myristoyl L-a-lysophosphatidylcholine; or
b. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-linoleoylphosphatidyl-choline; or
c. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-linoleoylphosphatidyl-choline, and myristoyl L-a-lysophosphatidylcholine; or
d. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and myristoyl L-a-lysophosphatidylcholine; or
e. linolenic acid, eicosapentaenoic acid, 1-octadecanoyl-sn-glycero-3-phosphoethanolamine, myristoyl L-a-lysophosphatidylcholine, 1-o-hexadecyl-2-lyso-glycero-3-phosphorylcholine, and 1-linoleoylphosphatidyl-choline; or
f. linolenic acid, eicosapentaenoic acid, myristoyl L-a-lysophosphatidylcholine, 3,6,7,12 alpha-tetrahydroxy-5beta-cholest-24-en-26-oic acid, 1-o-hexadecyl-2-lyso-glycero-3-phosphorylcholine, 1-o-hexadecyl-2-lyso-glycero-3-phosphorylcholine, and 1-linoleoylphosphatidyl-choline; or
g. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-hexadecyl-2-acetyl-glycero-3-phosphocholine; or
h. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, and 1-o-hexadecyl-2-lyso-glycero-3-phosphorylcholine; or
i. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, or;
j. linolenic acid, eicosapentaenoic acid, 1-octadecanoyl-sn-glycero-3-phosphoethanolamine, and myristoyl L-a-lysophosphatidylcholine; or
k. linolenic acid, eicosapentaenoic acid, myristoyl L-a-lysophosphatidylcholine, and 3,6,7,12 alpha-tetrahydroxy-5beta-cholest-24-en-26-oic acid; or
l. linolenic acid, eicosapentaenoic acid, and 1-octadecanoyl-sn-glycero-3-phosphoethanolamine.

In general, the panel that is used in the method comprises metabolite species that have been identified by at least one of the methods selected from nuclear magnetic resonance (NMR) spectroscopy, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In certain embodiments, the panel comprises metabolite species that have been identified by nuclear magnetic resonance (NMR) spectroscopy. In some embodiments, the panel comprises metabolite species that have been identified by liquid chromatography-mass spectrometry (LC-MS). Typically, the biofluid is selected from the group consisting of blood, plasma, serum, sweat, saliva, sputum, and urine. In preferred embodiments, the biofluid is serum.

In other aspects, a panel of metabolite species is disclosed that comprises a plurality of metabolite species selected from the group consisting of glutamine, histidine, isoleucine, threonine, 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine, and mixtures thereof. In certain embodiments, the panel comprises one to five metabolite species selected from the group consisting of glutamine, histidine, isoleucine, threonine, linolenic acid and mixtures thereof. In some embodiments, the panel comprises glutamine, isoleucine, threonine, and linolenic acid. In other embodiments, the panel comprises linolenic acid.

Further embodiments provide a panel of metabolic species consisting of components that have been selected from a population of metabolite species that have been shown to significantly distinguish a first outcome from a second outcome. Exemplary panels include, but are not limited to:
a. glutamine, histidine, isoleucine, threonine, and linolenic acid, or
b. glutamine, histidine, isoleucine, and threonine, or
c. glutamine, isoleucine, and threonine; or
d. glutamine, isoleucine, threonine, and linolenic acid; or
e. linolenic acid.

Other embodiments provide a panel of metabolic species consisting of components that have been selected from a population of metabolite species that have been shown to significantly distinguish a first outcome from a second outcome. Exemplary panels include, but are not limited to:
a. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, 1-linoleoylphosphatidyl-choline, 1-hexadecyl-2-acetyl-glycero-3-phosphocholine, 2-amino-3-methyl-1-butanol, and myristoyl L-a-lysophosphatidylcholine; or
b. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-linoleoylphosphatidyl-choline; or
c. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-linoleoylphosphatidyl-choline, and myristoyl L-a-lysophosphatidylcholine; or
d. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and myristoyl L-a-lysophosphatidylcholine; or
e. linolenic acid, eicosapentaenoic acid, 1-octadecanoyl-sn-glycero-3-phosphoethanolamine, myristoyl L-a-lysophosphatidylcholine, 1-o-hexadecyl-2-lyso-glycero-3-phosphorylcholine, and 1-linoleoylphosphatidyl-choline; or
f. linolenic acid, eicosapentaenoic acid, myristoyl L-a-lysophosphatidylcholine, 3,6,7,12 alpha-tetrahydroxy-5beta-cholest-24-en-26-oic acid, 1-o-hexadecyl-2-lyso-glycero-3-phosphorylcholine, 1-o-hexadecyl-2-lyso-glycero-3-phosphorylcholine, and 1-linoleoylphosphatidyl-choline; or
g. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-hexadecyl-2-acetyl-glycero-3-phosphocholine; or
h. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, and 1-o-hexadecyl-2-lyso-glycero-3-phosphorylcholine; or
i. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, or;
j. linolenic acid, eicosapentaenoic acid, 1-octadecanoyl-sn-glycero-3-phosphoethanolamine, and myristoyl L-a-lysophosphatidylcholine; or
k. linolenic acid, eicosapentaenoic acid, myristoyl L-a-lysophosphatidylcholine, and 3,6,7,12 alpha-tetrahydroxy-5beta-cholest-24-en-26-oic acid; or
l. linolenic acid, eicosapentaenoic acid, and 1-octadecanoyl-sn-glycero-3-phosphoethanolamine.

In other embodiments, a diagnostic cassette comprising such a panel of metabolic species is disclosed. In some embodiments, a diagnostic cassette comprises reagents for the detection of the metabolite species of such a panel.

Also disclosed is a kit for the analysis of a sample of a biofluid of a subject, comprising vessels containing aliquots of standards of each compound of a panel of metabolite species; a vessel containing an aliquot of an internal standard; and a vessel containing an aliquot of a control biofluid. Typically the control biofluid is serum from a control source that is conspecific with the subject. In certain embodiments, the panel comprises one to thirteen metabolite species selected from the group consisting of glutamine, histidine, isoleucine, threonine, 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine, and mixtures thereof. In some embodiments, the panel comprises glutamine, isoleucine, threonine, and linolenic acid. Typically, the kit includes instructions for use.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, in which corresponding reference characters indicate corresponding parts throughout the several views.

FIG. 1A shows a partial least squares (PLS) score plot based on 27 metabolite species detected by NMR spectroscopy for samples from all of the patients, with a stable disease outlier (filed triangle) in the lower right quadrant. Key: pathologic complete response (pCR, filled diamonds), stable disease (SD, filled triangles) and partial response (PR, open squares). FIG. 1B shows the partial least squares (PLS) score plot of FIG. 1A with the outlier removed. Key as in FIG. 1A. FIG. 1C shows a partial least squares (PLS) score plot based on based on nine lipid and fatty acid metabolite biomarkers detected by LC-MS for samples from all of the patients.

FIG. 2A-2E show individual box-and-whisker plots comparing the levels of five metabolite species in biofluid samples from patients assigned to the three different clinicopathological groups (pCR, PR and SD): (FIG. 2A) threonine; (FIG. 2B) glutamine; (FIG. 2C) isoleucine; (FIG. 2D) histidine; and (FIG. 2E) linolenic acid.

FIG. 3A-FIG. 3E show plots of the ROC curves for each of five metabolic biomarkers comparing samples from the three clinicopathological groups: the pCR population compared to the SD population, the pCR population compared to the combination of the PR population and the SD population. The left column shows ROC curves from PLS-DA models generated from the analysis of biofluid samples from subjects in the pathologic complete response population (pCR, N=8) and the stable disease population (SD). The right column shows ROC curves from PLS-DA models generated from the analysis of biofluid samples from subjects in the pathologic complete response population (pCR, N=8) and the combination of the PR population and the SD population (PR+SD, N=20). FIG. 3A, threonine, pCR vs. SD, AUROC=0.85, pCR vs. PR+SD, AUROC=0.61. FIG. 3B, glutamine, pCR vs. SD, AUROC=0.80, pCR vs. PR+SD, AUROC=0.64. FIG. 3C, isoleucine, pCR vs. SD, AUROC=0.83, pCR vs. PR+SD, AUROC=0.77. FIG. 3D, histidine, pCR vs. SD, AUROC=0.65, pCR vs. PR+SD, AUROC=0.79. FIG. 3E, linolenic acid, pCR vs. SD, AUROC=1, pCR vs. PR+SD, AUROC=0.86.

FIG. 4A is a plot of prediction results for the PLS-DA model based on three amino acid metabolite biomarkers (threonine, isoleucine and glutamine) detected by NMR spectroscopy, showing pathologic complete response (pCR, filled diamonds), stable disease (SD, filled triangles) and partial response (PR, open squares). Samples are ordered by patient number in their respective groups according to Table 2. FIG. 4B is a plot of the receptor operating curve (ROC curve) for a comparison of pathologic complete response (pCR, filled diamonds) vs stable disease (SD) using the cross-validated predicted class values (AUROC=0.81); FIG. 4C is a ROC curve for a comparison of samples from patients showing pathologic complete response vs samples from patients in the other groups, using the cross-validated predicted class values (AUROC=0.72).

FIG. 5A shows prediction results for the PLS-DA model based on combining isoleucine, glutamine and threonine, detected by NMR spectroscopy, and linolenic acid, detected by LC-MS, showing pathologic complete response (pCR, filled diamonds), stable disease (SD, filled triangles) and partial response (PR, open squares). Samples are ordered by patient number in their respective groups according to Table 2. FIG. 5B is a plot of the ROC curve for pathologic complete response (pCR) vs stable disease (SD) using the cross-validated predicted class values (AUROC=0.95); FIG. 5C is a plot of the ROC curve for pathologic complete response vs the other two groups combined using the cross-validated predicted class values (AUROC=0.89).

### DETAILED DESCRIPTION OF EXEMPLARY EMBODIMENTS

Breast cancer is a clinically heterogeneous disease, which necessitates a variety of treatments and leads to different outcomes. Because of this heterogeneity, only some women will benefit from preoperative chemotherapy. Identifying patients who will respond to chemotherapy and thereby improve their long-term survival has important implications to treatment protocols and outcomes, while identifying non responders may enable these patients to avail themselves of other investigational approaches or other potentially effective treatments.

In this study, serum metabolite profiling was performed to identify potential metabolic biomarker candidates that can predict response to neoadjuvant (preoperative) chemotherapy for breast cancer. Metabolic profiles of serum from patients classified on clinicopathological criteria as having a pathologic complete response (pCR, N=8), a partial response (N=14) and no response (referred to as “stable disease,” or SD, N=6) to preoperative chemotherapy were studied using a combination of nuclear magnetic resonance (NMR) spectroscopy, liquid chromatography-mass spectrometry (LC-MS) and statistical analysis methods.

Several partial least squares models were generated using metabolite concentration data from several panels of metabolite species to predict the assignment of samples from patents into the appropriate population of pCR, PR or SD. A prediction model based on the concentrations of four metabolic biomarkers, three amino acids (threonine, isoleucine, glutamine) identified using NMR spectroscopy, and linolenic acid, identified using LC-MS, correctly identified 80% of the patients whose tumors did not show pathologic complete response to chemotherapy.

Unless defined otherwise, all technical and scientific terms used herein have the meaning commonly understood by a person skilled in the art to which this invention belongs. Numbers in scientific notation are expressed as product of a coefficient between 1 and 10 and ten raised to an integer power (e.g., 9.6×10⁻⁴), or abbreviated as the coefficient followed by “E,” followed by the exponent (e.g., 9.6E-04).

As used herein, “metabolite” refers to any substance produced or used during all the physical and chemical processes within the body that create and use energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g. metabolite, metabolic precursor). The term “metabolite species” as used herein refers to an identified molecule or an identified molecular moiety, such as a lipid alkyl moiety, that is detectable by the measurement technique that is used. See U.S. published patent application US 2007/0221835, the contents of which are hereby incorporated by reference.

As used herein, “biological sample” refers to a sample obtained from a subject. In preferred embodiments, biological sample can be selected, without limitation, from the group of biological fluids (“biofluids”) consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, non-cellular portion of the blood, as distinguished from the serum, which is obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female. “Conspecific” means of or belonging to the same species, and when used as a noun, a member of the same species.

As used herein, “normal control subjects” or “normal controls” means healthy subjects who are clinically free of cancer. “Normal control sample” or “control sample” refers to a sample of biofluid that has been obtained from a normal control subject. A normal control sample or a control sample is preferably obtained from a conspecific of the test subject.

As used herein, “detecting” refers to methods which include identifying the presence or absence of substance(s) in the sample, quantifying the amount of substance(s) in the sample, and/or qualifying the type of substance.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a,” “an,” and “the” include plural referents unless the context clearly indicates otherwise. The terms “comprises,” “comprising,” and the like are intended to have the broad meaning ascribed to them in U.S. Patent Law and can mean “includes,” “including” and the like.

Metabolite profiling uses high-throughput analytical methods such as nuclear magnetic resonance spectroscopy and mass spectroscopy for the quantitative analysis of hundreds of small molecules (less than ˜1000 Daltons) present in biological samples. Owing to the complexity of the metabolic profile, multivariate statistical methods are extensively used for data analysis. The high sensitivity of metabolite profiles to even subtle stimuli can provide the means to detect the early onset of various biological perturbations in real time.

While some of these metabolite profiles were discovered using LC-MS methods, one of ordinary skill in the art will recognize that these identified biomarkers can be detected by alternative methods of suitable sensitivity, such as HPLC, immunoassays, enzymatic assays or clinical chemistry methods.

In one embodiment of the invention, samples may be collected from individuals over a longitudinal period of time. Obtaining numerous samples from an individual over a period of time can be used to verify results from earlier detections and/or to identify an alteration in marker pattern as a result of, for example, pathology. In preferred embodiments, the present disclosure provides methods for predicting the effectiveness of preoperative chemotherapy for breast cancer treatment.

In one embodiment of the invention, the samples are analyzed without additional preparation and/or separation procedures. In another embodiment of the invention, sample preparation and/or separation can involve, without limitation, any of the following procedures, depending on the type of sample collected and/or types of metabolic products searched: removal of high abundance polypeptides or proteins (e.g., albumin, and transferrin); addition of preservatives and calibrants, desalting of samples; concentration of sample substances; protein digestions; and fraction collection. In yet another embodiment of the invention, sample preparation techniques concentrate information-rich metabolic products and deplete polypeptides and proteins or other substances that would carry little or no information such as those that are highly abundant in serum.

In another embodiment of the invention, sample preparation takes place in a manifold or preparation/separation device. Such a preparation/separation device may, for example, be a microfluidics device, such as a diagnostic cassette. In yet another embodiment of the invention, the preparation/separation device interfaces directly or indirectly with a detection device. Such a preparation/separation device may, for example, be a fluidics device.

In another embodiment of the invention, the removal of undesired polypeptides (e.g., high abundance, uninformative, or undetectable polypeptides) can be achieved using high affinity reagents, high molecular weight filters, column purification, ultracentrifugation and/or electrodialysis. High affinity reagents include antibodies that selectively bind to high abundance polypeptides or reagents that have a specific pH, ionic value, or detergent strength. High molecular weight filters include membranes that separate molecules on the basis of size and molecular weight. Such filters may further employ reverse osmosis, nanofiltration, ultrafiltration and microfiltration.

Ultracentrifugation constitutes another method for removing undesired polypeptides. Ultracentrifugation is the centrifugation of a sample at about 60,000 rpm while monitoring with an optical system the sedimentation (or lack thereof) of particles. Finally, electrodialysis is an electromembrane process in which ions are transported through ion permeable membranes from one solution to another under the influence of a potential gradient. Since the membranes used in electrodialysis have the ability to selectively transport ions having positive or negative charge and reject ions of the opposite charge, electrodialysis is useful for concentration, removal, or separation of electrolytes.

In another embodiment of the invention, the manifold or microfluidics device or diagnostic cassette performs electrodialysis to remove high molecular weight polypeptides or undesired polypeptides. Electrodialysis can be used first to allow only molecules under approximately 35-30 kD to pass through into a second chamber. A second membrane with a very small molecular weight cutoff (roughly 500 Da) allows smaller molecules to exit the second chamber.

Upon preparation of the samples, metabolic products of interest may be separated in another embodiment of the invention. Separation can take place in the same location as the preparation or in another location. In one embodiment of the invention, separation occurs in the same microfluidics device where preparation occurs, but in a different location on the device. Samples can be removed from an initial manifold location to a microfluidics device or diagnostic cassette using various means, including an electric field. In another embodiment of the invention, the samples are concentrated during their migration to the microfluidics device or diagnostic cassette using reverse phase beads and an organic solvent elution such as 50% methanol. This elutes the molecules into a channel or a well on a separation device of a microfluidics device or diagnostic cassette.

Chromatography constitutes another method for separating subsets of substances. Chromatography is based on the differential absorption and elution of different substances. Liquid chromatography (LC), for example, involves the use of fluid carrier over a non-mobile phase. Conventional LC columns have an inner diameter of roughly 4.6 mm and a flow rate of roughly 1 ml/min. Micro-LC has an inner diameter of about 1.0 mm and a flow rate of about 40 μL/min. Capillary LC utilizes a capillary with an inner diameter of roughly 300 μm and a flow rate of approximately 5 μL/min. Nano-LC is available with an inner diameter of 50 μm-1 mm and flow rates of 200 nL/min. The sensitivity of nano-LC as compared to HPLC is approximately 3700 fold. Other types of chromatography suitable for additional embodiments of the invention include, without limitation, thin-layer chromatography (TLC), reverse-phase chromatography, high-performance liquid chromatography (HPLC), and gas chromatography (GC).

In another embodiment of the invention, the samples are separated using capillary electrophoresis separation. This will separate the molecules based on their electrophoretic mobility at a given pH (or hydrophobicity). In another embodiment of the invention, sample preparation and separation are combined using microfluidics technology. A microfluidic device is a device that can transport liquids including various reagents such as analytes and elutions between different locations using microchannel structures.

Suitable detection methods are those that have a sensitivity for the detection of an analyte in a biofluid sample of at least 50 μM. In certain embodiments, the sensitivity of the detection method is at least 1 μM. In other embodiments, the sensitivity of the detection method is at least 1 nM.

In one embodiment of the invention, the sample may be delivered directly to the detection device without preparation and/or separation beforehand. In another embodiment of the invention, once prepared and/or separated, the metabolic products are delivered to a detection device, which detects them in a sample. In another embodiment of the invention, metabolic products in elutions or solutions are delivered to a detection device by electrospray ionization (ESI). In yet another embodiment of the invention, nanospray ionization (NSI) is used. Nanospray ionization is a miniaturized version of ESI and provides low detection limits using extremely limited volumes of sample fluid.

In another embodiment of the invention, separated metabolic products are directed down a channel that leads to an electrospray ionization emitter, which is built into a microfluidic device (an integrated ESI microfluidic device). Such integrated ESI microfluidic device may provide the detection device with samples at flow rates and complexity levels that are optimal for detection. Furthermore, a microfluidic device may be aligned with a detection device for optimal sample capture.

Suitable detection devices can be any device or experimental methodology that is able to detect metabolic product presence and/or level, including, without limitation, IR (infrared spectroscopy), NMR (nuclear magnetic resonance spectroscopy), including variations such as correlation spectroscopy (COS_{y}), nuclear Overhauser effect spectroscopy (NOESY), and rotating frame nuclear Overhauser effect spectroscopy (ROESY), and Fourier Transform, 2-D PAGE technology, Western blot technology, tryptic mapping, in vitro biological assay, immunological analysis, LC-MS (liquid chromatography-mass spectrometry), LC-TOF-MS, LC-QTOF, LC-MS/MS, and MS (mass spectrometry).

For analysis relying on the application of NMR spectroscopy, the spectroscopy may be practiced as one-, two-, or multidimensional NMR spectroscopy or by other NMR spectroscopic examining techniques, among others also coupled with chromatographic methods (for example, as LC-NMR). In addition to the determination of the metabolic product in question, ¹H-NMR spectroscopy offers the possibility of determining further metabolic products in the same investigative run. Combining the evaluation of a plurality of metabolic products in one investigative run can be employed for so-called “pattern recognition”. Typically, the strength of evaluations and conclusions that are based on a profile of selected metabolite species, i.e., a panel of identified biomarkers, is improved compared to the isolated determination of the concentration of a single metabolite.

For immunological analysis, for example, the use of immunological reagents (e.g. antibodies), generally in conjunction with other chemical and/or immunological reagents, induces reactions or provides reaction products which then permit detection and measurement of the whole group, a subgroup or a subspecies of the metabolic product(s) of interest. Suitable immunological detection methods with high selectivity and high sensitivity (10-1000 pg, or 0.02-2 pmoles), e.g., Baldo, B. A., et al. 1991, A Specific, Sensitive and High-Capacity Immunoassay for PAF, Lipids 26(12): 1136-1139), that are capable of detecting 0.5-21 ng/ml of an analyte in a biofluid sample (Cooney, S. J., et al., Quantitation by Radioimmunoassay of PAF in Human Saliva), Lipids 26(12): 1140-1143).

In one embodiment of the invention, mass spectrometry is relied upon to detect metabolic products present in a given sample. In another embodiment of the invention, an ESI-MS detection device is relied upon to detect metabolic products present in a given sample. Such an ESI-MS may utilize a time-of-flight (TOF) mass spectrometry system. Quadrupole mass spectrometry, ion trap mass spectrometry, and Fourier transform ion cyclotron resonance (FTICR-MS) are likewise contemplated in additional embodiments of the invention.

In another embodiment of the invention, the detection device interfaces with a separation/preparation device or microfluidic device, which allows for quick assaying of many, if not all, of the metabolic products in a sample. A mass spectrometer may be utilized that will accept a continuous sample stream for analysis and provide high sensitivity throughout the detection process (e.g., an ESI-MS). In another embodiment of the invention, a mass spectrometer interfaces with one or more electrosprays, two or more electrosprays, three or more electrosprays or four or more electrosprays. Such electrosprays can originate from a single or multiple microfluidic devices.

In another embodiment of the invention, the detection system utilized allows for the capture and measurement of most or all of the metabolic products introduced into the detection device. In another embodiment of the invention, the detection system allows for the detection of change in a defined combination (“profile,” “panel,” “ensemble, or “composite”) of metabolic products.

Profiles of metabolite species in blood serum were constructed using NMR spectroscopy, LC-MS, and statistical analysis methods. The metabolite biomarkers discovered were selected to build a predictive model that was then used to test the classification accuracy.

Chemicals. For NMR experiments, deuterium oxide (D₂O, 99.9% D) was purchased from Cambridge Isotope Laboratories, Inc. (Andover, Mass.). Sodium azide (NaN₃) and the sodium salt of trimethylsilylpropionic acid-d4 (TSP) were purchased from Sigma-Aldrich (Milwaukee, Wis.). For LC-MS experiments, two internal standards (tridecanoic acid and chlorophenylalanine) and linolenic acid were purchased from Sigma-Aldrich (analytical grade, St. Louis, Mo.). HPLC-grade methanol and acetic acid were purchased from Fisher Scientific (Pittsburgh, Pa.).

Patients and serum samples. The patients were enrolled into the study between 2005 and 2008 (Table 1 and Table 2, below), and were treated according to current guidelines for neoadjuvant therapy. Those patients with locally advanced breast cancer and eligible for neoadjuvant chemotherapy were enrolled in this study. Table 3, below, summarizes the inclusion and exclusion criteria for the protocol. All patients were recruited and treated at the Department for Obstetrics and Gynecology, University of Tuebingen. Collection of data. Patient's study data were collected in case report forms (CRF); a CRF was completed for each included patient. Documents which identify the patient (e.g. the patient identification log and the signed informed consent) were maintained in confidence by the investigator.

Data processing and ownership of data. Recorded data of the patients were made anonymous before being transmitted. Completeness and plausibility was checked and data were transferred into an electronic data processing system by the KKS-UKT gGmbH. A double data entry was performed with a double data check.

Archiving. To comply with legal requirements, the Sponsor retains records for 10 years after finishing this study or premature termination. The source documents in the “Interdisziplinäres Brustzentrum des Universitätsklinikums Tübingen” are archived in accordance with national regulations.

TABLE 1
Summary of clinicopathological characteristics
of the breast cancer patients studied.

| | | Pathologic | | |
|---|---|---|---|---|
| | | Complete | Partial | Stable |
| Patient | | Response | Response | Disease |
| Characteristics | | (pCR) | (PR) | (SD) |
| Number of Patients | | 8 | 14 | 6 |
| Average Age (range) | | 49.5 (37-60) | 48.7 (34-64) | 43.7 (37-59) |
| Stage | T1 | 0 | 0 | 2 |
| | T2 | 6 | 10 | 3 |
| | T3 | 2 | 1 | 1 |
| | T4 | 0 | 3 | 0 |
| | N0 | 6 | 1 | 2 |
| | N1 | 1 | 4 | 2 |
| | N+ | 1 | 9 | 0 |
| | NX | 0 | 0 | 2 |
| | M0 | 8 | 12 | 5 |
| | MX | 0 | 2 | 1 |
| Menopause | pre | 4 | 7 | 4 |
| Status | post | 4 | 6 | 1 |
| | N/A | 0 | 1 | 1 |
| ER Status | pos | 3 | 12 | 4 |
| | neg | 5 | 2 | 2 |
| Partial | pos | 5 | 11 | 5 |
| Response | neg | 3 | 3 | 1 |
| Status | | | | |
| Her2/neu | pos | 5 | 5 | 3 |
| Status | neg | 3 | 9 | 3 |
| Grading | G1 | 0 | 0 | 1 |
| | G2 | 6 | 10 | 5 |
| | G2-3 | 1 | 0 | 0 |
| | G3 | 1 | 4 | 0 |

TABLE 2
Clinicopathological characteristics of the breast cancer patients studied.

| Patient | | | | TNM after | Menopause | ER | PR | Her2/neu | |
|---|---|---|---|---|---|---|---|---|---|
| No. | Status | Age | Stage | PST | Status | Status | Status | Status | Grading |
| 1 | pCR | 37 | T3N0M0 | ypTispN0M0 | pre | neg | neg | pos | G2 |
| 2 | pCR | 60 | T2N + M0 | ypT0pN0M0 | post | neg | pos | pos | G2 |
| 3 | pCR | 49 | T3N0M0 | ypT0pN0M0 | pre | pos | pos | neg | G2 |
| 4 | pCR | 49 | T2N0M0 | ypT0pN0M0 | pre | neg | pos | neg | G2 |
| 5 | pCR | 37 | T2N1M0 | ypT0pN0M0 | pre | neg | pos | neg | G2 |
| 6 | pCR | 56 | T2N0M0 | ypT0pN0M0 | post | pos | pos | pos | G2-3 |
| 7 | pCR | 56 | T2N0M0 | ypT0N0M0 | post | pos | neg | pos | G2 |
| 8 | pCR | 52 | T2N0M0 | ypT0pN0M0 | post | neg | neg | pos | G3 |
| 9 | PR | 41 | T4bN + MX | ypT1bpN0M0 | pre | pos | pos | neg | G2 |
| 10 | PR | 64 | T4N + MX | ypT2pN3aM0 | post | pos | pos | neg | G2 |
| 11 | PR | 62 | T2N + M0 | T1cN0MX | post | pos | neg | neg | G2 |
| 12 | PR | 59 | T4bN + M0 | ypT2pN3aM0 | post | pos | pos | neg | G2 |
| 13 | PR | 35 | T2N1M0 | ypT1cpN0M0 | pre | neg | neg | pos | G3 |
| 14 | PR | 42 | T3N + M0 | ypT2pN1M0 | pre | pos | pos | neg | G2 |
| 15 | PR | 38 | T2N0M0 | ypT1apN0M0 | pre | neg | pos | neg | G2 |
| 16 | PR | 44 | T2N + M0 | ypTispN0M0 | pre | pos | pos | pos | G3 |
| 17 | PR | 50 | T2N + M0 | ypT1bpN1mi | post | pos | pos | neg | G2 |
| | | | | M0 | | | | | |
| 18 | PR | 55 | T2N + M0 | ypT1bpN0M0 | N/A | pos | neg | neg | G3 |
| 19 | PR | 34 | T2N1M0 | ypT1bpN0M0 | pre | pos | pos | pos | G3 |
| 20 | PR | 49 | T2N1M0 | ypT1cpN1M0 | pre | pos | pos | neg | G2 |
| 21 | PR | 51 | T2N + M0 | ypT1apN0M0 | post | pos | pos | pos | G2 |
| 22 | PR | 58 | T2N1M0 | ypT1cpN1aM0 | post | pos | pos | pos | G2 |
| 23 | SD | 40 | T2NXMX | ypT2pN2aM0 | N/A | pos | pos | neg | G2 |
| 24 | SD | 39 | T3N0M0 | ypT3pN0M0 | pre | pos | pos | neg | G2 |
| 25 | SD | 37 | T2N1M0 | ypT2pN1M0 | pre | pos | pos | neg | G1 |
| 26 | SD | 59 | T1N1M0 | ypT3pN2a M0 | post | pos | pos | pos | G2 |
| 27 | SD | 38 | T2N0M0 | ypT2pN0M0 | pre | neg | pos | pos | G2 |
| 28 | SD | 49 | T1cNXM0 | ypT1cpN0M0 | pre | neg | neg | pos | G2 |

pCR: pathologically complete response;
PR: partial response;
SD: stable disease,
ER estrogen receptor;
PR: progesterone receptor

TABLE 3
Inclusion and Exclusion Criteria

| | |
|---|---|
| Study | Women with newly diagnosed breast cancer, |
| population: | age ≧18 years. |
| Indication: | Patients with locally advanced breast cancer |
| | eligible for neoadjuvant chemotherapy. |
| Criteria for | Signed informed consent and willingness to |
| inclusion | comply with requirements of the clinical |
| | investigational plan (CIP). |
| | Female |
| | Age ≧18 years |
| | Histopathologically verified lesions of locally |
| | advanced breast cancer (T2-4, N0-1, M0) |
| | Indication to neoadjuvant chemotherapy |
| | Patient consent to neoadjuvant chemotherapy |
| | Negative result of a serum human chorionic |
| | gonadotropin pregnancy test before start of the |
| | study for women of childbearing potential |
| | Ability to consent to study participation |
| Criteria for | Any known allergy to contrast agent |
| exclusion | Patients suffering from claustrophobia |
| | Patients with pacemakers, metallic cardiac |
| | valves, magnetic material such as surgical |
| | clips, implanted electronic perfusion pumps or |
| | any other conditions that would preclude |
| | proximity to a strong magnetic field |
| | Patient unwilling to cooperate |
| | Ongoing or previous (within the last 30 days) |
| | participation in a second clinical study with |
| | impacts on the present study |
| | Pregnancy or nursing |
| | Contraindications of neoadjuvant chemotherapy |

The response to neoadjuvant chemotherapy in patients with locally advanced breast cancer was based on the correlation of several clinicopathological assessments: magnetic resonance imaging (MRI), two dimensional (2D)/three dimensional (3D) ultrasound (US) and mammography (MG) with histopathology (study code BCD-001, funded by the BMBF (German Federal Ministry of Education and Research). Based on their response, patients were categorized as having pathologic complete response (pCR), partial response (PR) or stable disease (SD). Here, pathologic complete response is defined as the disappearance of all tumor deposits; partial response indicates a reduction of tumor volume exceeding 50%; while tumor reduction less than 50% or increase of volume up to 25% is scored as stable disease (stable disease). The response of the tumors to the neoadjuvant chemotherapy was evaluated pathologically by classifying the regressive changes using a semiquantitative scoring system from 0 to 4 (0=no effect, 1=resorption and tumor sclerosis, 2=minimal residual invasive tumor [<0.5 cm], 3=residual noninvasive tumor only, 4=no tumor detectable) according to the tumor regression grading described by Sinn et al. (Sinn, H. P., et al., 1994. Histologic regression of breast cancer after primary (neoadjuvant) chemotherapy. Geburtshilfe Frauenheilkd. 54, 552-558.) A consultant pathologist, blinded to clinical outcome, reviewed all paired biopsy and surgical specimens.

All patients in this study consented to neoadjuvant chemotherapy and signed the informed consent. Samples were de-identified in compliance with a protocol approved by the institutional review boards (IRB committees) at both Eberhard-Karls-University of Tuebingen and Purdue University.

Serum samples from all 28 breast cancer patients with baseline samples available before preoperative chemotherapy were obtained. Clinical and histopathological data are listed in Table 1 and Table 2, above. Of these patients, eight belonged to pathologic complete response group, six to stable disease group and fourteen to partial response group. Blood (2×8 mL) was drawn 45 minutes before the start of the chemotherapy. The collected blood was allowed to clot for 45 minutes at room temperature and centrifuged for 10 minutes at 3,000 rpm at room temperature; the upper serum phase was then isolated, aliquoted and frozen at −80° C. until further use.

Patients were treated according to the design of GeparQuattro. GeparQuattro is a neoadjuvant trial, exploring the effect of simultaneous or sequential use of capecitabine (“X,” pentyl[1-(3,4-dihydroxy-5-methyltetrahydrofuran-2-yl)-5-fluoro-2-oxo-1H-pyrimidin-4-yl]carbamate) with docetaxel (“Doc,” 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}) after 4 cycles of epirubicin/cyclophosphamide (EC) as well as simultaneous trastuzumab on pathologic complete response of previously untreated stage I-III breast cancers. Treatment included 4 three weekly EC cycles consisting of epirubicin ((8R,10S)-10-((2S,4S,5R,6S)-4-amino-5-hydroxy-6-methyltetrahydro-2H-pyran-2-yl)-6,8,11-trihydroxy-8-(2-hydroxyacetyl)-1-methoxy-7,8,9,10-tetrahydrotetracene-5,12-dione, 90 mg/m²), with simultaneous administration of cyclophosphamide ((RS)—N,N-bis(2-chloroethyl)-1,3,2-oxazaphosphinan-2-amine 2-oxide, 600 mg/m²). Subsequently, patients received docetaxel (100 mg/m²) in three weekly intervals. Patients with Her2/neu positive tumors were also treated with Trastuzumab (6 mg/kg, i.v.) every three weeks, starting with a loading dose of 8 mg/kg i.v. on day 1 of the first EC-cycle.

### Example 1

Predictions of Models Based on Normalized NMR Metabolite Species and Nine Metabolite Species Identified Using LC-MS

NMR spectroscopy. Frozen serum samples were thawed and 530 μL was mixed with 5 μL 0.1% NaN₃ solution. Sixty μL of a 0.5 mM TSP solution was used as an internal standard. ¹H 1D NMR experiments were performed using the CPMG (Carr-Purcell-Meiboom-Gill) pulse sequence coupled with water presaturation on a Bruker Avance-500 spectrometer equipped with a TXI gradient cryoprobe. One hundred and twenty eight scans with 16 k time domain data points were collected using a spectral width of 6,000 Hz. An exponential weighting function corresponding to 1.0 Hz line broadening was applied to the time domain data before Fourier transformation. The spectra were then phased, baseline corrected and referenced to alanine (6=1.48 ppm) using Bruker Topspin 3.0 software.

Liquid chromatography resolved mass spectrometry (LC-MS). Frozen serum samples (100 μL) were thawed, and protein was precipitated by adding 200 μL methanol containing two internal standards, tridecanoic acid and chlorophenylalanine, used to monitor extraction efficiency. The solutions were centrifuged at 13,200 rpm for 30 minutes; the resulting supernatants were dried under vacuum and reconstituted in 25 μL methanol/water (1:1). A pooled sample, which was a mixture of small random volumes from all 28 samples, was extracted using the same procedure as above. This sample was used as a quality control (QC) and was analyzed after every ten patient samples. LC-MS analysis was performed using an Agilent 6520 HPLC-QTOF system (Agilent Technologies, Santa Clara, Calif.) consisting of an Agilent 1200 SL liquid chromatograph coupled with a time-of-flight (TOF) mass spectrometer. The reconstituted serum samples were gradient-eluted at 600 μL/min using (A) 0.2% acetic acid in water and (B) 0.2% acetic acid in methanol (2% B to 98% B in 13 min, 98% B for 6 min). A 3 μL sample aliquot was injected onto a 2.1×50 mm Agilent Zorbax Extend-C18 1.8 μm particle column with a 2.1×30 mm Agilent Zorbax SB-C8 3.5 μm particle guard column heated to 60° C. Electrospray ionization (ESI) was used in positive mode. The MS interface capillary was maintained at 325° C., with a sheath gas flow of 9 L/min. The spray voltage for positive ion injection was 4.0 kV. The instrument was scanned over a range of 50-1000 m/z. Agilent MassHunter Workstation LC-TOF and QTOF Acquisition software (B.02.01) were used for automatic peak detection and mass spectrum deconvolution.

Data pre-processing. From the ¹H NMR spectra, 27 metabolite species, i.e., spectral regions with metabolite peaks, were identified and integrated after local baseline correction, and normalized to the total sum of all the metabolites. In order to identify distinguishing metabolite biomarkers, the integral for each metabolite species was statistically compared using the Student's t-test between the three different populations (pCR, PR and SD) of patients.

The LC-MS data were processed using Agilent's MassHunter Qualitative Analysis software (version B.03.01). A list of ion intensities for each peak detected was generated, matching m/z and retention time (RT) for each ion. Agilent MassHunter Workstation Mass Profiler Professional software (version B.02.00) was then used for compound peak alignment and removal of any peaks with missing values (ion intensity=1) in more than one sample from any group; 115 metabolite species passed this filter. Internal standard peaks were also removed. Finally, the Agilent Formula Database (Agilent 2010) was used for compound identification by matching the accurate mass spectrum to a database of metabolite compounds. The Student's t-test was performed between pathologic complete response and stable disease samples, and 9 metabolite species with low p-values (<0.1) (Table 4, below) were selected as biomarker candidates for further statistical analysis.

TABLE 4
Summary of LC-MS compounds having low p-values (<0.1).

| | | Mass | Mass | RT | p-value | p-value | p-value |
|---|---|---|---|---|---|---|---|
| | | Detected | Calculated | Detected | (pCR vs | (pCR vs | (PR vs |
| Compound Name | Formula | (Da) | (Da) | (min) | SD) | PR) | SD) |
| 1-Linoleoylphosphatidyl- | C₂₆H₅₀NO₇P | 519.3319 | 519.3325 | 11.81 | 0.01 | 0.98 | 0.01 |
| choline | | | | | | | |
| Myristoyl L-a- | C₂₂H₄₆NO₇P | 467.3009 | 467.3012 | 11.46 | 0.04 | 0.16 | 0.48 |
| lysophosphatidylcholine | | | | | | | |
| 1-Hexadecyl-2-acetyl- | C₂₆H₅₄NO₇P | 523.3632 | 523.3638 | 12.60 | 0.04 | 0.12 | 0.99 |
| glycero-3-phosphocholine | | | | | | | |
| 3,6,7,12 alpha-tetrahydroxy- | C₂₇H₄₄O₆ | 464.3118 | 464.3138 | 13.65 | 0.05 | 0.61 | 0.01 |
| 5beta-cholest-24-en-26-oic | | | | | | | |
| acid* | | | | | | | |
| Eicosapentaenoic acid | C₂₀H₃₀O₂ | 302.225 | 302.2246 | 11.78 | 0.0007 | 0.0002 | 0.44 |
| 1-O-hexadecyl-2-lyso- | C₂₄H₅₂NO₆P | 481.3528 | 481.3532 | 12.50 | 0.07 | 0.06 | 0.64 |
| glycero-3- | | | | | | | |
| phosphorylcholine | | | | | | | |
| linolenic acid** | C₁₈H₃₀O₂ | 278.2247 | 278.2246 | 11.44 | 0.03 | 0.0002 | 0.32 |
| 2-Amino-3-methyl-1- | C₅H₁₃NO | 103.0998 | 103.0997 | 12.32 | 0.09 | 0.64 | 0.13 |
| butanol | | | | | | | |
| GPEtn(18:0/0:0) | C₂₃H₄₈NO₇P | 481.3166 | 481.3168 | 12.47 | 0.007 | 0.66 | 0.01 |

*Compound found upregulated in pCR samples.
**Validated using a pure compound.
GPEtn(18:0/0:0) = 1-octadecanoyl-sn-glycero-3-phosphoethanolamine

Statistical Analysis. The peak integrals from all 27 metabolite species that were identified by NMR spectroscopy and nine metabolite species that were identified by LC-MS with p<0.1 listed in Table 4, above, were imported into Matlab software (Mathworks) installed with the PLS toolbox (Eigenvector Research, Inc., version 4.0) for partial least squares (PLS) analysis to obtain clustering information and identify outliers. The same software was also used for partial least squares discriminant analysis (PLS-DA) modeling. Leave-one-out cross validation (CV) was chosen, and the number of latent variables were selected according to the root mean square error of CV procedure. The R statistical package (version 2.8.0) was used to generate receiver operating characteristics (ROC) curves and box-and-whisker plots.

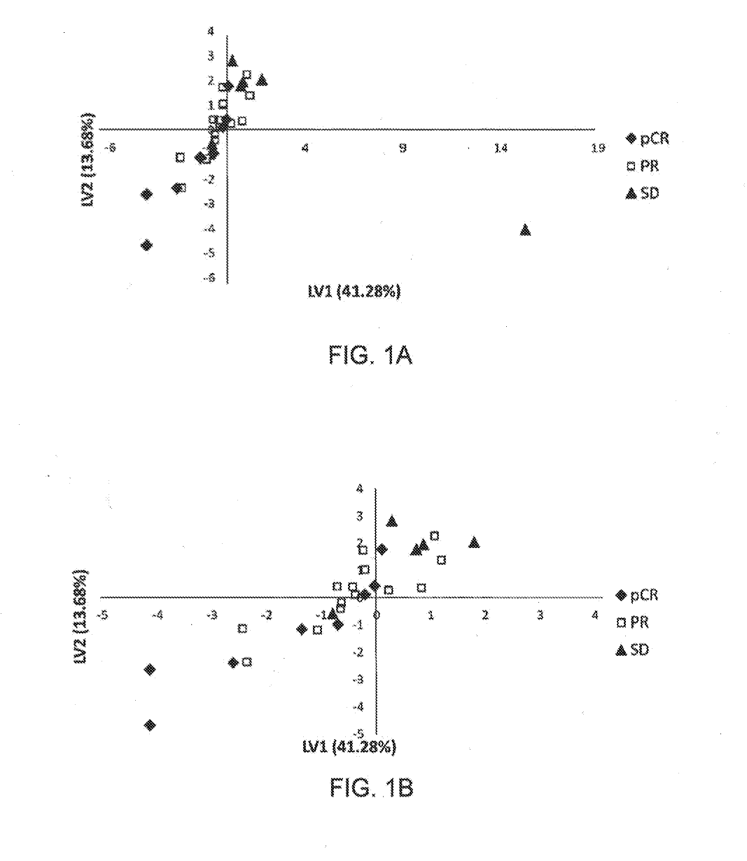

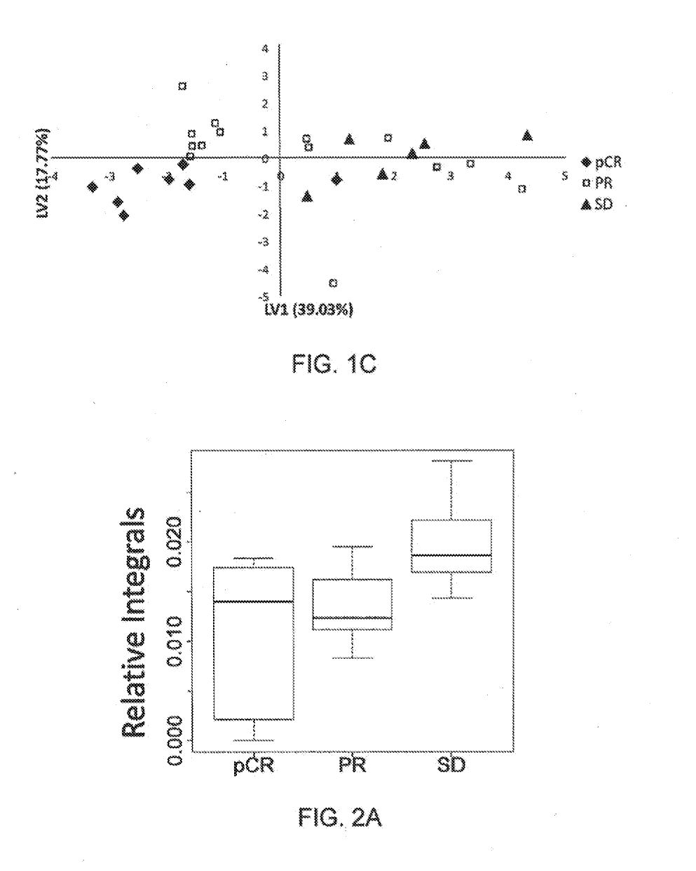

Results. In order to visualize the intrinsic grouping of samples and identify outliers, PLS analysis was performed using the 27 metabolite species identified by NMR spectroscopy, and the results of this analysis are shown in FIG. 1A. The results showed that the stable disease patient group has one outlier in the lower right quadrant (filled triangle) that was omitted from further analysis. The new PLS score plot obtained after removing this outlier is shown in FIG. 1B. Both pathologic complete response (pCR) and stable disease (SD) groups were separated along both LV1 and LV2, while the partial response (PR) group lies between pathologic complete response and stable disease.

Similar analysis performed using the nine metabolite species with p<0.1 (Table 4) obtained from LC-MS data was also performed: 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine. The results are shown in FIG. 1C. As observed for the metabolite species identified using NMR spectroscopy metabolite species identified using LC-MS also separated pathologic complete response from the stable disease group of patients. However, the clustering for the partial response group was not as well defined.

As shown in Table 4, above, all of the listed compounds except 2-Amino-3-methyl-1-butanol could distinguish a sample drawn from a patient who would have an outcome of pathological complete recovery from sample drawn from a patient who would have an outcome of stable breast cancer disease at a level of p<0.05. Subsets of the complete panel of nine metabolic species can be selected that distinguish a first outcome from a second outcome at a level of p<0.05. Exemplary panels include, but are not limited to:
a. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, 1-linoleoylphosphatidyl-choline, 1-hexadecyl-2-acetyl-glycero-3-phosphocholine, 2-amino-3-methyl-1-butanol, and myristoyl L-a-lysophosphatidylcholine; or
b. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-linoleoylphosphatidyl-choline; or
c. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-linoleoylphosphatidyl-choline, and myristoyl L-a-lysophosphatidylcholine; or
d. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and myristoyl L-a-lysophosphatidylcholine; or
e. linolenic acid, eicosapentaenoic acid, 1-octadecanoyl-sn-glycero-3-phosphoethanolamine, myristoyl L-a-lysophosphatidylcholine, 1-o-hexadecyl-2-lyso-glycero-3-phosphorylcholine, and 1-linoleoylphosphatidyl-choline; or
f. linolenic acid, eicosapentaenoic acid, myristoyl L-a-lysophosphatidylcholine, 3,6,7,12 alpha-tetrahydroxy-5beta-cholest-24-en-26-oic acid, 1-o-hexadecyl-2-lyso-glycero-3-phosphorylcholine, 1-o-hexadecyl-2-lyso-glycero-3-phosphorylcholine, and 1-linoleoylphosphatidyl-choline; or
g. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-hexadecyl-2-acetyl-glycero-3-phosphocholine; or
h. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, and 1-o-hexadecyl-2-lyso-glycero-3-phosphorylcholine; or
i. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, or;
j. linolenic acid, eicosapentaenoic acid, 1-octadecanoyl-sn-glycero-3-phosphoethanolamine, and myristoyl L-a-lysophosphatidylcholine; or
k. linolenic acid, eicosapentaenoic acid, myristoyl L-a-lysophosphatidylcholine, and 3,6,7,12 alpha-tetrahydroxy-5beta-cholest-24-en-26-oic acid; or
l. linolenic acid, eicosapentaenoic acid, and 1-octadecanoyl-sn-glycero-3-phosphoethanolamine.

TABLE 5
Summary of NMR metabolite species having low p-values.

| | | | p-value | p-value | |
|---|---|---|---|---|---|
| | | | (pathologic | pathologic | p-value |
| | | | complete | complete | (partial |
| chemical | | | response | response | response |
| shift | multi- | | vs stable | vs partial | vs stable |
| (δ) | plicity | assignment | disease) | response) | disease) |
| 4.24 | m | threonine | 0.04 | 0.28 | 0.01 |
| 1.00 | d | isoleucine | 0.04 | 0.10 | 0.20 |
| 2.09 | m | glutamine | 0.01 | 0.30 | 0.02 |
| 7.07 | d | histidine | 0.29 | 0.01 | 0.54 |

### Example 2

### Predictions of Models Based on Four Amino Acid Metabolite Species and One Lipid Metabolite Species

Comparison of the data for the four amino acid metabolite species that were identified with NMR samples from patients in different clinicopathological groups using the Student's t-test showed comparisons based on the four metabolite species to be statistically significant (p<0.05) (Table 5, above). One-way ANOVA was not used here since the objective is to see the difference between each pair of groups. The p-values indicate that levels of three metabolite species, isoleucine, threonine and glutamine, were significantly different in samples from patients in the pathologic complete response group and the stable disease group. The levels of two metabolite species, threonine and glutamine, were significantly different in samples from patients in the pathologic complete response group and the partial group. The levels of only one metabolite, histidine, differed significantly between pathologic complete response and partial response.

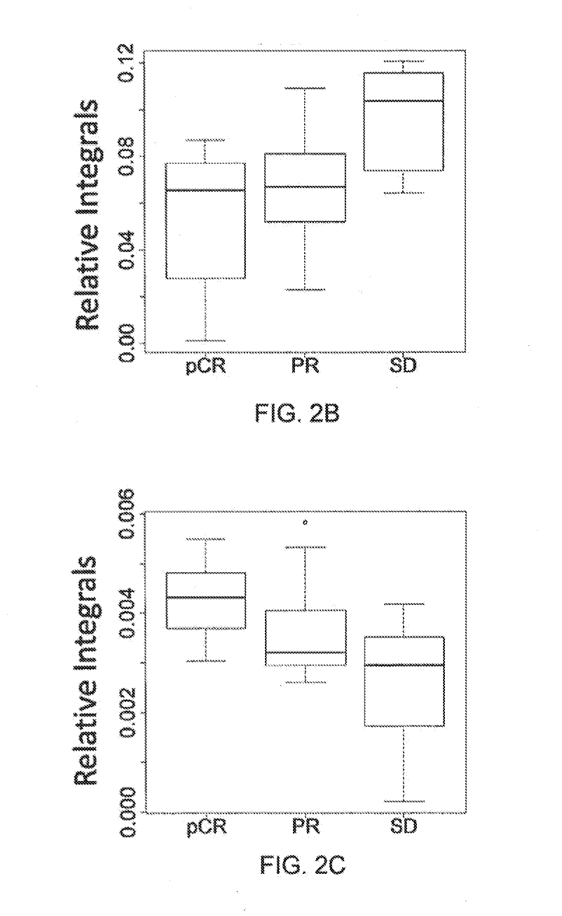

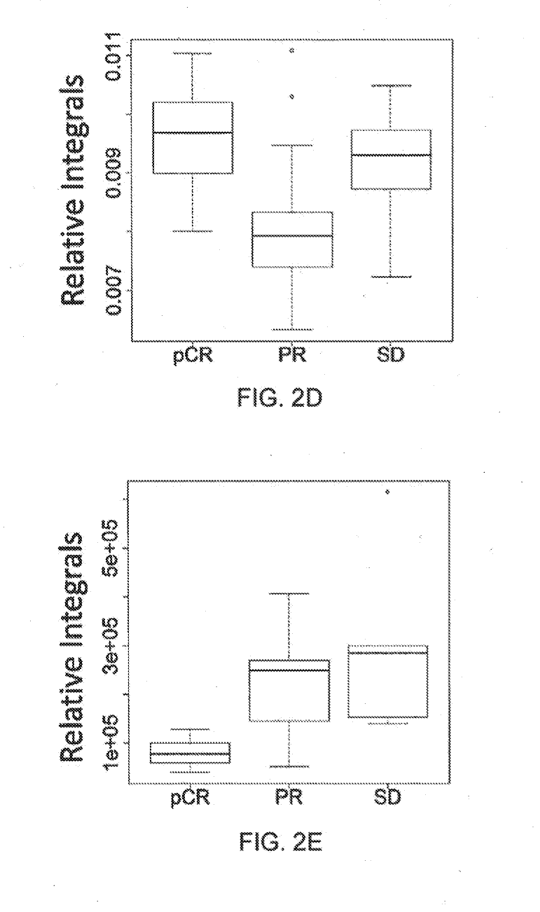

The LC-MS data showed that the most statistically differentiating compounds found were long-chain lipids. The most interesting of these, linolenic acid, was validated using a pure, commercially obtained compound. This metabolite species separated pathologic complete response from stable disease samples perfectly, which makes combining linolenic acid with other LC-MS detected markers unnecessary for model building. Statistical analysis shows linolenic acid to be significantly different between pathologic complete response and stable disease groups (p<0.01). Performance of these metabolite species was evaluated individually using receiver operative characteristic (ROC) curves and box-and-whisker plots. FIG. 2A-2E show individual box-and-whisker plots comparing the levels of five metabolite species in biofluid samples from patients assigned to the three different clinicopathological groups (pCR, PR and SD): (FIG. 2A) threonine; (FIG. 2B) glutamine; (FIG. 2C) isoleucine; (FIG. 2D) histidine; and (FIG. 2E) linolenic acid.

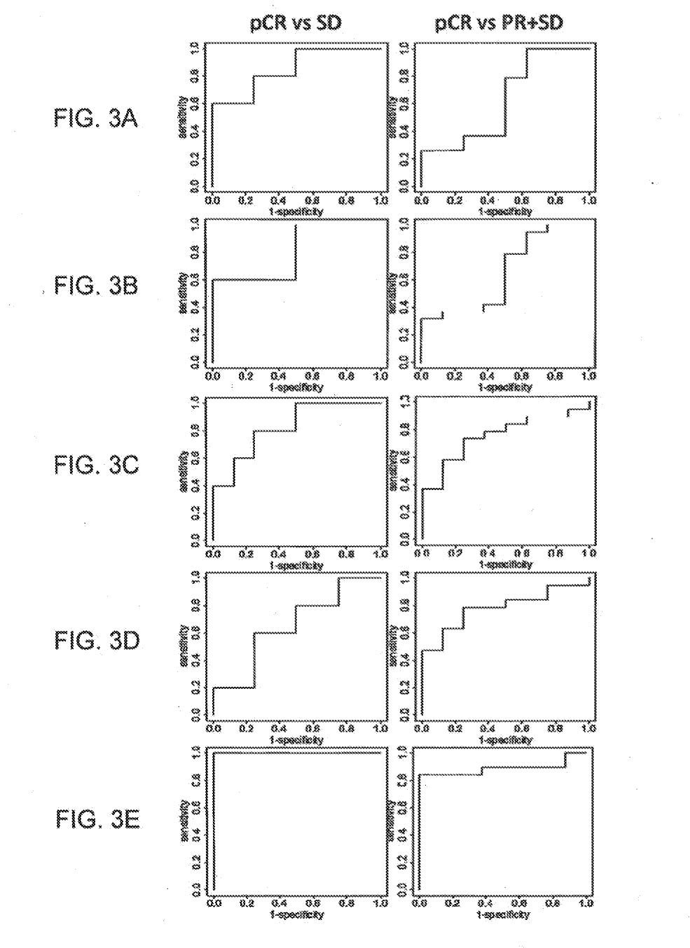

FIG. 3A-FIG. 3E show plots of the ROC curves for each of five metabolic biomarkers comparing the pCR population to the SD population or comparing the pCR population to the combination of the PR population and the SD population. The left column shows ROC curves from PLS-DA models generated from the analysis of biofluid samples from subjects in the pathologic complete response population (pCR, N=8) and the stable disease population (SD). The right column shows ROC curves from PLS-DA models generated from the analysis of biofluid samples from subjects in the pathologic complete response population (pCR, N=8) and the combination of the PR population and the SD population (PR+SD, N=20). FIG. 3A, threonine, pCR vs. SD, AUROC=0.85, pCR vs. RP+SD, AUROC=0.61. FIG. 3B, glutamine, pCR vs. SD, AUROC=0.80, pCR vs. RP+SD, AUROC=0.64. FIG. 3C, isoleucine, pCR vs. SD, AUROC=0.83, pCR vs. RP+SD, AUROC=0.77. FIG. 3D, histidine, pCR vs. SD, AUROC=0.65, pCR vs. RP+SD, AUROC=0.79. FIG. 3E, linolenic acid, pCR vs. SD, AUROC=1, pCR vs. RP+SD, AUROC=0.86.

The concentration distribution for all of the metabolite biomarkers except histidine showed a consistent trend along the spectrum from pathologic complete response to partial response to stable disease. The concentration of the metabolic biomarkers threonine, glutamine and linolenic acid increased, but isoleucine decreased, along the spectrum from pathologic complete response to partial response to stable disease. The four metabolite biomarkers, threonine, glutamine, isoleucine and linolenic acid each showed a minimum AUROC of 0.80 in classifying pathologic complete response and stable disease patient. Only linolenic acid distinguished pathologic complete response from the combined population of stable disease and partial response patients with an AUROC of greater than 0.80 (FIG. 3E). As mentioned above, linolenic acid distinctly separated pathologic complete response from stable disease with an AUROC=1.

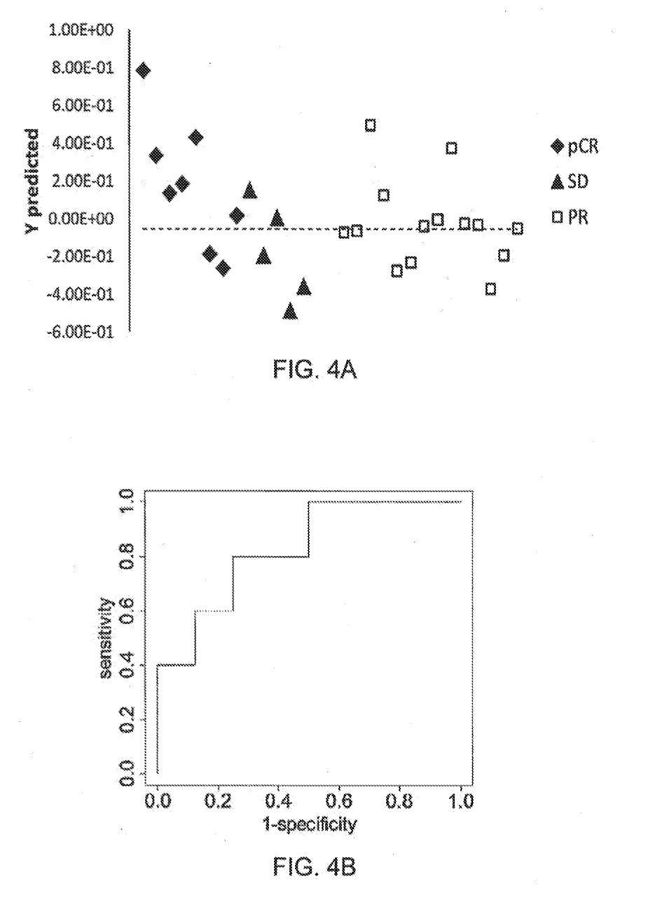

Further analysis focused on evaluating the performance of the metabolite species in combination. First, we combined the data from the three metabolic biomarkers identified using NMR spectroscopy, threonine, glutamine and isoleucine, that distinguished pathologic complete response and stable disease, performed PLS-DA and built a statistical classification model. Leave-one-out cross validation was used to reduce over-fitting and estimate model accuracy. FIG. 4A-4C show prediction results using this model for different patient groups. The AUROC for distinguishing pathologic complete response and stable disease was 0.81. However, as with the individual metabolite species, the performance for distinguishing pathologic complete response from the other two groups, partial response and stable disease, was relatively poor (AUROC=0.72).

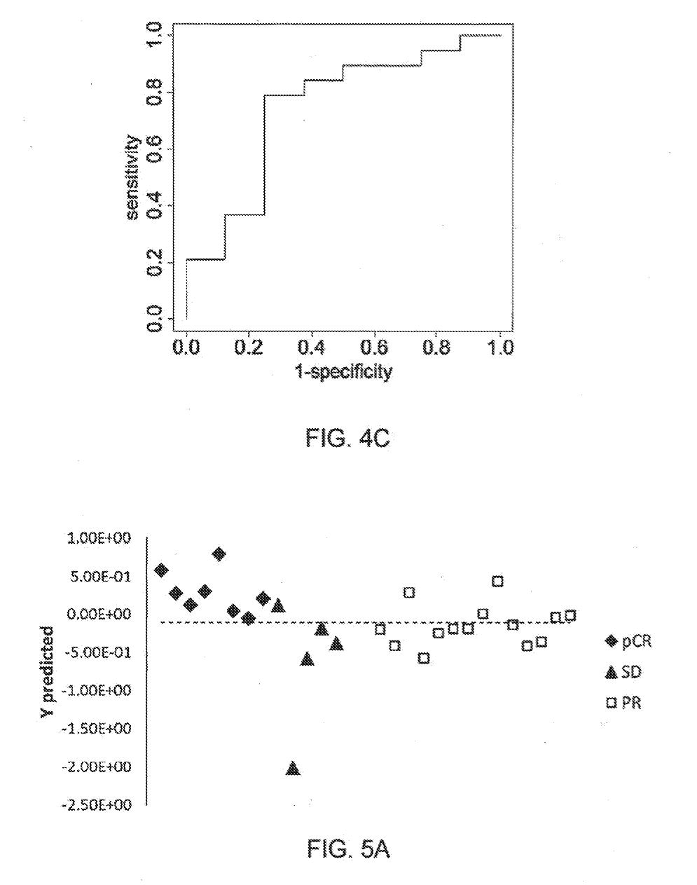

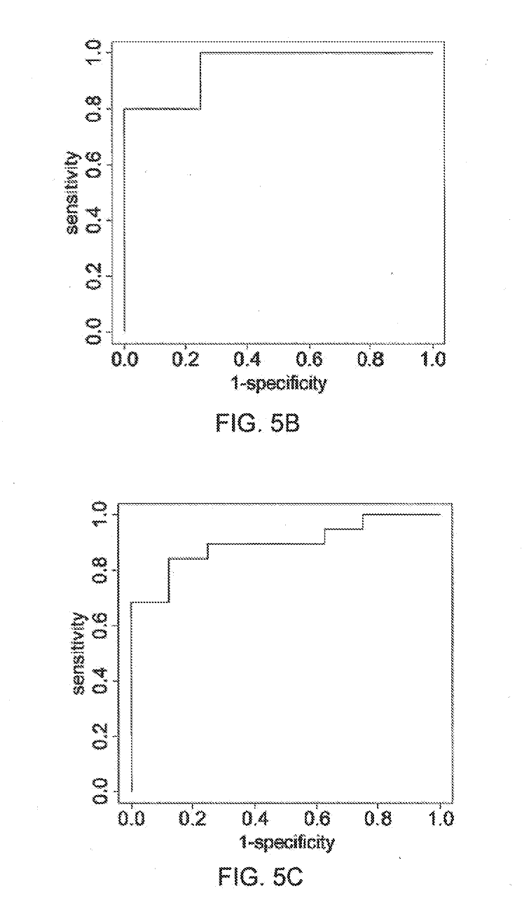

Analysis was also performed combining the data from analysis for three metabolic biomarkers, threonine, glutamine and isoleucine that were identified using NMR, with the data from analysis for linolenic acid, which was detected using LC-MS. The results from the statistical analysis are shown in FIG. 5A-5C. The model shows 100% selectivity and 80% sensitivity for the prediction of pathologic complete response vs stable disease with an AUROC of 0.95, which is significantly better compared to the classification provided by the three marker model presented in FIG. 4A-4C. Samples from patients in the partial response population were not classified well by either model.

### Example 3

### Effect of Tissue Markers on Metabolite Levels

Although the clinical and histopathological parameters of the patients and tumors in each response group are quite heterogeneous, the serum samples still show distinct clusters. In order to investigate the impact of different subtypes of breast cancer on the performance of four markers we used for model building, p-values from the Student's t-test were obtained by comparing samples from different ER; PR; Her2/neu status for pCR, PR, SD. All of these sample data are summarized in Table 6, above. No p-value less than 0.05 can be found, indicating that none of the four markers is significantly altered by any of these parameters in the data set.

TABLE 6
Summary of p-Values (<0.1) for Four Markers from the Student's
t-test by comparing different ER, PR, or Her2/neu groups

| | | | | |
|---|---|---|---|---|
| | | | | linolenic |
| Metabolite: | threonine | glutamine | isoleucine | acid |

ER− vs ER+

| | | | | |
|---|---|---|---|---|
| pCR | 0.16 | 0.13 | 0.13 | 0.24 |
| PR | 0.05 | 0.30 | 0.48 | 0.78 |
| SD | 0.17 | 0.07 | 0.82 | 0.51 |
| All samples | 0.58 | 0.90 | 0.34 | 0.13 |

PR− vs PR+

| pCR | 0.87 | 0.83 | 0.94 | 0.90 |
|---|---|---|---|---|
| PR | 0.96 | 0.58 | 0.42 | 0.91 |
| SD | N/A* | | | |
| All samples | 0.92 | 0.68 | 0.32 | 0.61 |

Her2/neu− vs Her2/neu+

| | | | | |
|---|---|---|---|---|
| pCR | 0.75 | 0.59 | 0.56 | 0.69 |
| PR | 0.86 | 0.86 | 0.25 | 0.27 |
| SD | 0.26 | 0.47 | 0.09 | 0.67 |
| All samples | 0.62 | 0.42 | 0.15 | 0.17 |

*Only one sample available in the PR− group for SD patients.

Currently, tissue markers such as ER, Her2/neu and PR only provide limited predictive information about the results of neoadjuvant chemotherapy. For example it is known that neoadjuvant therapy is not very effective in ER+ women. In our data, only 3 of 7 ER+women showed pCR compared to SD. By comparison, our 4 marker panel was able to identify all 3 ER+women who showed pCR and all 4 of the ER+women with SD after neoadjuvant chemotherapy (FIG. 5A and FIG. 5B). See Table 7, below. ER− women did respond better to neoadjuvant chemotherapy in our patient cohort, where 5 of 7 showed pCR compared to SD. For a woman with ER− tissue marker status, according to our data she would have had a 71% (5/7) change of pCR vs SD. By comparison, we were able to identify all 5 ER− women who showed pCR and one of the two who showed SD (FIG. 5A and FIG. 5B).

TABLE 7

| | Prediction of pCR | | Prediction of SD | |
|---|---|---|---|---|
| | | Using 4 | | Using 4 |
| Tumor | Using Tumor | Metabolite | Using Tumor | Metabolite |
| Type | Marker | Panel | Marker | Panel |
| ER+ | 3/7 (43%) | 3/3 (100%) | 4/7 (57%) | 4/4 (100%) |
| ER− | 5/7 (71%) | 5/5 (100%) | 2/7 (29%) | 1/2 (50%) |
| Her2/neu+ | 5/8 (63%) | 5/5 (100%) | 3/8 (37%) | 2/2 (100%) |
| Her2/neu− | 3/6 (50%) | 3//3 (100%) | 3/6 (50%) | 2/3 (66%) |

Some women with Her2/neu positive tissue markers also respond to neoadjuvant. In our data, 5 of 8 Her2/neu positive women showed pCR compared to SD, while the 6 women with Her2/neu negative tissue markers were split evenly between pCR and SD (FIG. 5A and FIG. 5B). Again based on our 4 marker panel, we were able to correctly identify all 5 Her2/neu+ and 3 Her2/neu− patients who showed pCR. We correctly identified 2 out of 2 Her2/neu+ patients with SD (1 patient sample was eliminated as an outlier), and 2 of 3 Her2/neu− patients (FIG. 5A and FIG. 5B). Triple negative women (those with ER-, Her2/neu- and PR-tumors) also respond to neoadjuvant chemotherapy. However, we did not have any triple negative patients to evaluate our metabolite profile. Overall, our patient cohort was representative of the types of response rates that are commonly seen. And the 4 marker panel was able to identify women who showed pCR, with accuracies that exceeded the predictions based on tumor markers.

What is claimed is:

1. A method of forecasting a treatment outcome before subjecting a breast cancer patient to preoperative chemotherapy, comprising the steps of:
measuring the concentration of at least one metabolite species in a sample of a biofluid taken from a subject having breast cancer before preoperative chemotherapy treatment, wherein each metabolite species is a component of a panel of a plurality of metabolite species, wherein the components of the panel have been selected from a population of metabolite species that have been shown to significantly distinguish a first outcome from a second outcome;
wherein a statistical model has been constructed using the combined measured concentration differences of each component in the panel of a plurality of metabolite species in a first sample from a subject having the first outcome and the measured concentration of each component of the panel of a plurality of metabolite species in a second sample from a subject having the second outcome, wherein the statistical model distinguishes the first sample from the second sample;
using the statistical model to determine the relationship of the concentrations of the components of the panel of metabolite species measured in the test sample to the concentrations of the components of the panel of metabolite species that are characteristic of the first outcome and characteristic of the second outcome;
thereby forecasting an outcome of subjecting a breast cancer patient to preoperative chemotherapy.

2. The method of claim 1 wherein the step of measuring the concentrations further includes measuring the concentration of at least one component of a panel of a plurality of metabolite species in a sample of a biofluid from a control source, wherein the metabolite species is a component of a panel of a plurality of metabolite species; and
wherein the statistical model is a partial least squares model.

3. The method of claim 1 wherein the outcomes being determined are:
a. the first outcome is pathological complete recovery and the second outcome is stable disease, or
b. the first outcome is pathological complete recovery and the second outcome is partial response, or
c. the first outcome is stable disease and the second outcome is partial response; or
d. the first outcome is pathological complete recovery and the second outcome is stable disease plus partial response.

4. The method of claim 1 wherein the panel of metabolite species comprises one to thirteen compounds selected from the group consisting of glutamine, histidine, isoleucine, threonine, 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine, and mixtures thereof.

5. The method of claim 1 wherein the panel comprises two to five metabolite species selected from the group consisting of glutamine, histidine, isoleucine, threonine, linolenic acid and mixtures thereof.

6. The panel of claim 4 wherein the panel is selected from the group consisting of:
a. glutamine, histidine, isoleucine, threonine, and linolenic acid, or
b. glutamine, histidine, isoleucine, and threonine, or
c. glutamine, isoleucine, and threonine; or
d. glutamine, isoleucine, threonine, and linolenic acid; or
e. linolenic acid.

7. The method of claim 4 wherein the panel is selected from the group consisting of:
a. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, 1-linoleoylphosphatidyl-choline, 1-hexadecyl-2-acetyl-glycero-3-phosphocholine, 2-amino-3-methyl-1-butanol, and myristoyl L-a-lysophosphatidylcholine; or
b. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-linoleoylphosphatidyl-choline; or
c. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-linoleoylphosphatidyl-choline, and myristoyl L-a-lysophosphatidylcholine; or
d. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and myristoyl L-a-lysophosphatidylcholine; or
e. linolenic acid, eicosapentaenoic acid, 1-octadecanoyl-sn-glycero-3-phosphoethanolamine, myristoyl L-a-lysophosphatidylcholine, 1-o-hexadecyl-2-lyso-glycero-3-phosphorylcholine, and 1-linoleoylphosphatidyl-choline; or
f. linolenic acid, eicosapentaenoic acid, myristoyl L-a-lysophosphatidylcholine, 3,6,7,12 alpha-tetrahydroxy-5beta-cholest-24-en-26-oic acid, 1-o-hexadecyl-2-lyso-glycero-3-phosphorylcholine, 1-o-hexadecyl-2-lyso-glycero-3-phosphorylcholine, and 1-linoleoylphosphatidyl-choline; or
g. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-hexadecyl-2-acetyl-glycero-3-phosphocholine; or
h. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, and 1-o-hexadecyl-2-lyso-glycero-3-phosphorylcholine; or
i. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, or;
j. linolenic acid, eicosapentaenoic acid, 1-octadecanoyl-sn-glycero-3-phosphoethanolamine, and myristoyl L-a-lysophosphatidylcholine; or
k. linolenic acid, eicosapentaenoic acid, myristoyl L-a-lysophosphatidylcholine, and 3,6,7,12 alpha-tetrahydroxy-5beta-cholest-24-en-26-oic acid; or
l. linolenic acid, eicosapentaenoic acid, and 1-octadecanoyl-sn-glycero-3-phosphoethanolamine.

8. The method of claim 1 wherein the panel comprises metabolite species that have been identified by a plurality of methods selected from nuclear magnetic resonance (NMR) spectroscopy, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry.

9. The method of claim 1 wherein the panel comprises metabolite species that have been identified by nuclear magnetic resonance (NMR) spectroscopy.

10. The method of claim 1 wherein the panel comprises metabolite species that have been identified by liquid chromatography-mass spectrometry (LC-MS).

11. The method of claim 1, wherein the biofluid is selected from the group consisting of blood, plasma, serum, sweat, saliva, sputum, and urine.

12. The method of claim 1, wherein the biofluid is serum.

13. A panel of metabolite species comprising one to thirteen metabolite species selected from the group consisting of glutamine, histidine, isoleucine, threonine, 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine, and mixtures thereof.

14. The panel of claim 13 wherein the panel consists of two to five metabolite species selected from the group consisting of glutamine, histidine, isoleucine, threonine, linolenic acid and mixtures thereof.

15. The panel of claim 13 wherein the panel is selected from the group consisting of:
a. glutamine, histidine, isoleucine, threonine, and linolenic acid, or
b. glutamine, histidine, isoleucine, and threonine, or
c. glutamine, isoleucine, and threonine; or
d. glutamine, isoleucine, threonine, and linolenic acid; or
e. linolenic acid.

16. The panel of claim 13 wherein the panel is selected from the group consisting of:
a. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, 1-linoleoylphosphatidyl-choline, 1-hexadecyl-2-acetyl-glycero-3-phosphocholine, 2-amino-3-methyl-1-butanol, and myristoyl L-a-lysophosphatidylcholine; or
b. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-linoleoylphosphatidyl-choline; or
c. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-linoleoylphosphatidyl-choline, and myristoyl L-a-lysophosphatidylcholine; or
d. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and myristoyl L-a-lysophosphatidylcholine; or
e. linolenic acid, eicosapentaenoic acid, 1-octadecanoyl-sn-glycero-3-phosphoethanolamine, myristoyl L-a-lysophosphatidylcholine, 1-o-hexadecyl-2-lyso-glycero-3-phosphorylcholine, and 1-linoleoylphosphatidyl-choline; or
f. linolenic acid, eicosapentaenoic acid, myristoyl L-a-lysophosphatidylcholine, 3,6,7,12 alpha-tetrahydroxy-5beta-cholest-24-en-26-oic acid, 1-o-hexadecyl-2-lyso-glycero-3-phosphorylcholine, 1-o-hexadecyl-2-lyso-glycero-3-phosphorylcholine, and 1-linoleoylphosphatidyl-choline; or
g. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, 1-o-hexadecyl-2-lyso-glycero-3-phosphorylcholine, and 1-hexadecyl-2-acetyl-glycero-3-phosphocholine; or
h. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, and 1-o-hexadecyl-2-lyso-glycero-3-phosphorylcholine; or
i. linolenic acid, eicosapentaenoic acid, 1-octadecanoyl-sn-glycero-3-phosphoethanolamine, 3,6,7,12 alpha-tetrahydroxy-5beta-cholest-24-en-26-oic acid, or;
j. linolenic acid, eicosapentaenoic acid, 1-octadecanoyl-sn-glycero-3-phosphoethanolamine, and myristoyl L-a-lysophosphatidylcholine; or
k. linolenic acid, eicosapentaenoic acid, myristoyl L-a-lysophosphatidylcholine, and 3,6,7,12 alpha-tetrahydroxy-5beta-cholest-24-en-26-oic acid; or
l. linolenic acid, eicosapentaenoic acid, and 1-octadecanoyl-sn-glycero-3-phosphoethanolamine.

17. A diagnostic cassette comprising the panel of claim 13.

18. A diagnostic cassette comprising reagents for the detection of the metabolite species of the panel of claim 13.

19. A kit for the analysis of a sample of a biofluid of a subject, comprising:
a. vessels containing aliquots of standards of each metabolite species of a panel of claim 13;
b. a vessel containing an aliquot of an internal standard; and
c. a vessel containing an aliquot of a control biofluid.

20. The kit of claim 19 wherein the control biofluid is serum from a control source that is conspecific with the subject.

21. The kit of claim 19 wherein the panel comprises two to thirteen metabolite species selected from the group consisting of glutamine, histidine, isoleucine, threonine, 1-linoleoylphosphatidyl-choline, myristoyl L-a-lysophosphatidylcholine, 1-hexadecyl-2-acetyl-glycero-3-phosphocholine, 3,6,7,12 alpha-tetrahydroxy-5beta-cholest-24-en-26-oic acid, eicosapentaenoic acid, 1-o-hexadecyl-2-lyso-glycero-3-phosphorylcholine, linolenic acid, 2-amino-3-methyl-1-butanol, and 1-octadecanoyl-sn-glycero-3-phosphoethanolamine, and mixtures thereof.

22. The kit of claim 19 wherein the panel is selected from the group consisting of:
a. glutamine, histidine, isoleucine, threonine, and linolenic acid, or
b. glutamine, histidine, isoleucine, and threonine, or
c. glutamine, isoleucine, and threonine; or
d. glutamine, isoleucine, threonine, and linolenic acid; or
e. linolenic acid.
